(19) United States
(12) Patent Application Publication
(10) Pub. No.: US 2008/0045872 A1
(43) Pub. Date: Feb. 21, 2008

(54) Elastic Bandage with Electrodes Spaced Apart From One Another

(75) Inventors: Hans B. Bauerfeind, Zeulenroda (DE); Holger Reinhardt, Kempen (DE); Rainer Scheuermann, Rainsdorf (DE)

Correspondence Address: BIRCH STEWART KOLASCH & BIRCH, PO BOX 747, FALLS CHURCH, VA, 22040-0747, US

(73) Assignee: Bauerfeind AG, Zeulenroda (DE)

(21) Appl. No.: 10/590,221

(22) PCT Filed: Feb. 23, 2005
(86) PCT No.: PCT/EP05/01941

§ 371 Date: May 22, 2007

(30) Foreign Application Priority Data
Feb. 25, 2004 (DE) 10 2004 009 210.9

(51) Int. Cl. A61H 1/00; A61N 1/00

(52) U.S. Cl. 602/19; 607/2; 607/48

(57) ABSTRACT

The invention relates to an elastic bandage comprising electrodes which are arranged at a distance from each other, and which are to be connected to an electric stimulation device. The elastic region of the bandage supporting the electrodes is covered, on the outer side thereof, by a pocket which is essentially less elastic than the elastic area, and which contains an elastic cushion as a support for the electrodes.

The invention relates to an elastic bandage with electrodes spaced apart from one another for connection to an electric stimulation device.

Such a bandage is described and disclosed in the WO document WO 01/02052 A2. In this document, various bandages are explained, each of which bears electrodes on its side facing the skin of the wearer. Said electrodes consist of knit-in lead wires and establish contact with the skin by touch. The wires are knit in by the means that for example, a knitting pattern is used which lends the lead wires the necessary longitudinal elasticity, or a zigzag stitch is used. The entire region with electrodes thus has the elasticity normally demanded of a bandage.

The object of the invention is to design an elastic bandage with the initially described design in which the effectiveness of the electrical stimulation is significantly increased. This is accomplished in accordance with the invention in that the elastic region of the bandage bearing the electrodes is covered on its outside by a pocket which is far less elastic than the elastic region and which contains a resilient cushion as support for the electrodes.

As a result of the aforementioned design of the region of the bandage that bears the electrodes, the contact pressure of the electrodes with respect to the skin is increased substantially as compared to the contact pressure of a normal elastic bandage, thus substantially increasing the conductivity of the points of contact between the electrodes and the skin. As a result of the essentially inelastic pocket, the side of the bandage bearing the electrodes is pushed away in a manner of speaking, since the elasticity of the cushion can only act in the direction toward the wearer's skin. The more tightly the bandage is drawn about the body part in question, the more firmly the cushion, which bears against the essentially inelastic pocket toward the outside, presses against the skin. This increased pressure on the skin has an especially beneficial medical effect because the muscle contractions resulting from the electrical stimulation are buffered by the resilient cushion, so that the electric stimulation device can have full scope for developing its effectiveness, especially in the dynamic range of use of the bandage. Naturally, this also applies to muscle contractions resulting from normal movements of the particular individual wearing the bandage.

In order to connect the stimulation device to the electrodes, it is useful for the stimulation device to be attached to the electrodes by means of lead wires, which are worked into the material of the bandage by designing a plurality of successive bending points. These bending points may achieve their form on account of a textile knitting or weaving process, but a zigzag stitch is also possible, for example.

In order to position the stimulation device as close as possible to the electrodes, it is useful to fasten the stimulation device to the bandage itself, and in a detachable manner, so that it also can be removed at any time.

There are numerous possibilities for the filling of the resilient cushion, for example, resilient foam. However, it is also possible to design the resilient cushion as an inflatable tube, which is then inserted in the pocket of the bandage.

Example embodiments of the invention are shown in the drawings, in which:

FIG. I shows a perspective view of a bandage for the back;

FIG. 2 shows the same bandage from the outside with a view of the pocket;

FIG. 3 shows a cross-section along line III-III from FIG. 2;

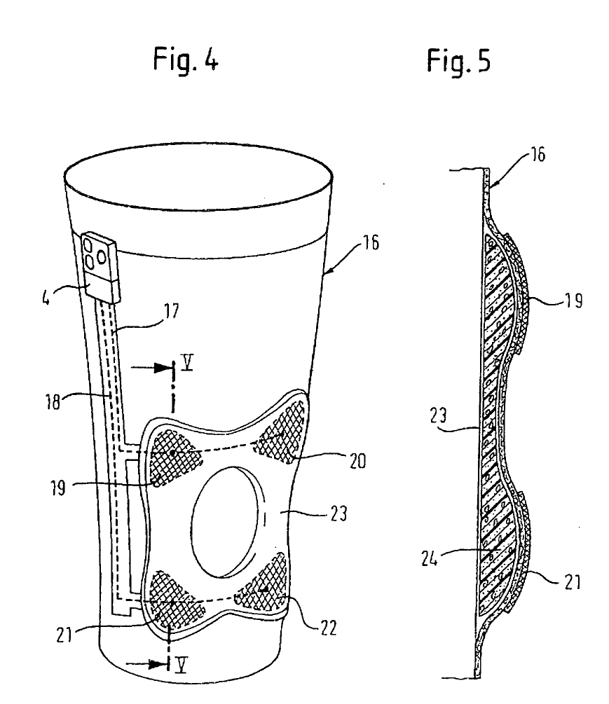

FIG. 4 shows a perspective view of a bandage for the knee joint;

FIG. 5 shows a cross-section along line V-V from FIG. 4.

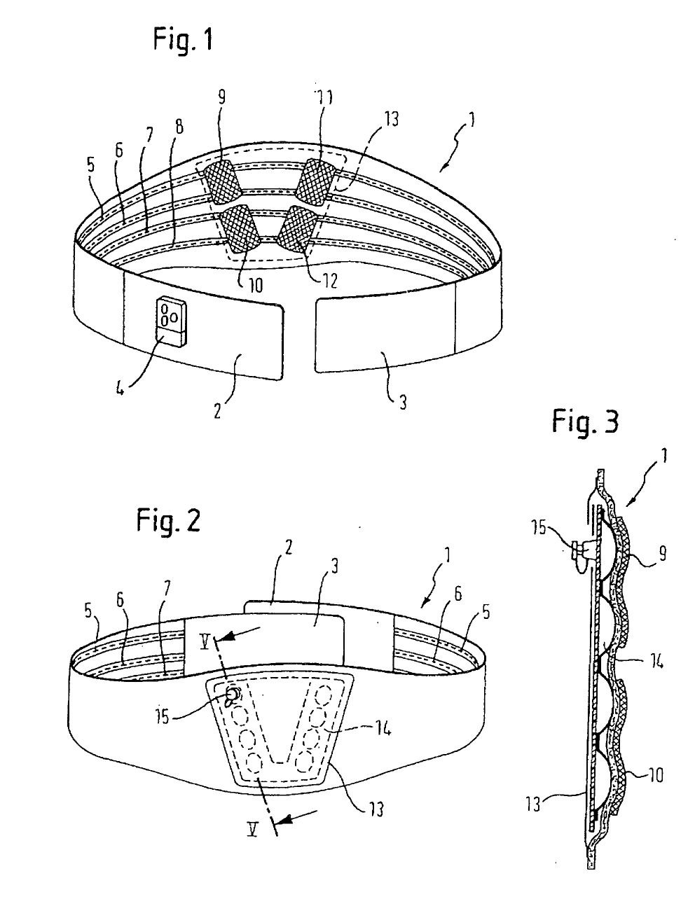

The bandage shown in FIG. 1 is a bandage for the back 1, which is closed with its two ends 2 and 3 on the stomach, the two ends being joined to one another in the customary way by means of hook-and-loop fastening. Attached to the outside of the part of the bandage for the back 1 facing the end 2 is the stimulation device 4, which is suspended by a loop of some sort, for example. Extending from the stimulation device 4 on the inside of the bandage for the back 1 are lead wires 5, 6, 7 and 8, which terminate in the electrodes 9, 10, 11 and 12 affixed to the inside of the bandage for the back 1. Affixed to the back of the bandage for the back 1, which is to say the side facing away from the electrodes 9 through 12, is the pocket 13, which is indicated in FIG. 1 by dashed lines and which is described in detail with reference to FIG. 2.

The supply of electricity to the electrodes 9 through 12 takes place here such that the electrodes 9 and 11 are supplied from one pole, and the electrodes 10 and 12 are supplied from the other pole, with the electrical impulses emitted by the stimulation device.

FIG. 2 shows the same bandage for the back 1, but here it is shown in perspective view in a position in which the two ends 2 and 3 overlap. Affixed to the back of the bandage for the back 1, is the pocket 13, which is made of a material with substantially less elasticity than the elasticity of the bandage for the back 1. As resilient cushion here, the pocket 13 contains the tube 14, which has a trapezoidal shape in keeping with the shape of the pocket 13, and thus fills the pocket 13 well. Attached in a known manner to the tube 14 is the valve 15, by which means the tube 14 can be inflated to any extent desired, resulting in the following effect: Since the pocket 13 cannot expand on account of its essential inelasticity, the expansion of the tube 14 forces the material of the bandage for the back 1 located over the pocket 13 to stretch in the direction toward the wearer's back, and thus press against the wearer's skin with a pressure adjusted in accordance with the degree of inflation of the tube 14. In this way, the contact resistance between the electrodes 9, 10, 11 and 12 (according to FIG. 1), which are thereby subjected to pressure, and the wearer's skin is lessened accordingly, by which means the intensity of the effects of the impulses emitted by the stimulation device can be adjusted accordingly.

Shown in FIG. 3 is a cross-section along line III-III from FIG. 2, showing the special design of the tube 14. The tube 14 is inflated by means of the valve 15, during which process the material of the bandage 1 pushes away from the pocket 13, thus pressing the electrodes 9 and 10 against the skin of the individual wearing the bandage.

Shown in FIG. 4 is another exemplary embodiment for the use of an electric stimulation device with a resilient cushion, involving a bandage for the knee 16. The stimulation device 4 is suspended in the region of the top edge of the knee bandage 16. The two lead wires 17 and 18 are routed from said device to the electrode, being routed in such a manner that the lead wire 17 leads to the two top electrodes 19 and 20, while the lead wire 18 is connected to the two bottom electrodes 21 and 22. Consequently, in this design a voltage drop is always produced in the longitudinal direction of the knee, which is to say from electrode 19 to electrode 21, and from electrode 20 to electrode 22. As in the exemplary embodiment shown in FIG. 1, the electrodes 19, 20, 21 and 22 are affixed to the inside of the knee bandage 16. Attached to the outside of the knee bandage in the vicinity of the electrodes 19 through 22 is the pocket 23, which functions in the same manner as the pocket 13 described in connection with FIGS. 1 and 2. In contrast to the exemplary embodiment shown in FIGS. I and 2, the pocket 23 here is filled with a resilient foam, which exerts the necessary pressure on the skin via the electrodes 19 and 22.

FIG. 5 shows a cross-section along the line V-V from FIG. 4, which shows the attachment of the pocket 23 to the material of the bandage 16. Inserted between the pocket 23 and the material of the bandage 16 is the resilient cushion 24 made of foam, which exerts the necessary pressure on the electrodes 19 and 21 attached to the material of the bandage 16. When the knee bandage 16 is applied, the electrodes 19 and 21 then press against the wearer's skin, thus conveying the effect of the electrical stimulation from the stimulation device 4.

Mention must be made of the fact that it is also possible to use, as the resilient material for the resilient cushion, other resilient materials whose properties make them capable of exerting pressure in a yielding way on the relevant electrodes.

1. Elastic bandage with electrodes spaced apart from one another for connection to an electric stimulation device, characterized in that the elastic region of the bandage bearing the electrodes is covered on its outside by a pocket which is far less elastic than the elastic region and which contains a resilient cushion as support for the electrodes attached to the inside of the bandage.

2. Bandage according to claim 1, characterized in that the stimulation device is attached to the electrodes by means of lead wires, which are worked into the material of the bandage by designing a plurality of successive bending points.

3. Bandage according to claim 1, characterized in that the stimulation device is fastened to the bandage in a detachable manner.

4. Bandage according to claim 1, characterized in that the resilient cushion is filled with resilient foam.

5. Bandage according to claim 1, characterized in that the resilient cushion consists of an inflatable tube.

6. Bandage according to claim 2, characterized in that the stimulation device is fastened to the bandage in a detachable manner.

7. Bandage according to claim 2, characterized in that the resilient cushion is filled with resilient foam.

8. Bandage according to claim 3, characterized in that the resilient cushion is filled with resilient foam.

9. Bandage according to claim 2, characterized in that the resilient cushion consists of an inflatable tube.

10. Bandage according to claim 3, characterized in that the resilient cushion consists of an inflatable tube.

11. Bandage according to claim 4, characterized in that the resilient cushion consists of an inflatable tube.
